(19) United States
(12) Patent Application Publication
(10) Pub. No.: US 2011/0021701 A1
(43) Pub. Date: Jan. 27, 2011

(54) MATERIAL COMPRISING MICROBIALLY SYNTHESIZED CELLULOSE ASSOCIATED WITH A SUPPORT LIKE A POLYMER AND/OR FIBRE

(75) Inventors: Alexander Bismarck, London (GB); Julasak Juntaro, London (GB); Athanasios Mantalaris, London (GB); Marion Pommet, London (GB); Milo S.P. Shaffer, London (GB)

Correspondence Address: CHOATE, HALL & STEWART LLP, TWO INTERNATIONAL PLACE, BOSTON, MA, 02110, US

(73) Assignee: IMPERIAL INNOVATIONS LIMITED, London (GB)

(21) Appl. No.: 12/377,329

(22) PCT Filed: Aug. 14, 2007
(86) PCT No.: PCT/GB2007/003081

§ 371 Date: Oct. 15, 2010

(30) Foreign Application Priority Data
Aug. 16, 2006 (GB) 0616290.3

(51) Int. Cl. C12P 19/04; C08G 63/46; C08B 3/06; C08B 3/08; C08B 1/00; C07K 1/04

(52) U.S. Cl. 525/54.23; 525/54.21; 536/69; 536/68; 536/56; 530/374; 530/373; 530/357; 435/101

(57) ABSTRACT

The invention relates to a material comprising cellulose in association with a support selected from a polymer and/or a fibre, wherein said cellulose is produced by a micro-organism. The cellulose-reinforced material is provided for incorporation into a composite.

The present invention relates to a reinforced material comprising cellulose in association with a support selected from a polymer or a fibre, wherein the cellulose is produced by a micro-organism. The invention further relates to processes for producing the material and composites comprising the material.

A composite is a structural product made of two or more distinct components. While each of the components remains physically distinct, composite materials exhibit a synergistic combination of the properties of each component, resulting in a material with extremely favourable and useful characteristics. Composites are generally composed of a matrix component and a reinforcement component. The reinforcement provides the special mechanical and/or physical properties of the material and is provided as fibres or fragments of material. The matrix surrounds and binds the fibres or fragments together to provide a material which is durable, stable to heat, stable to corrosion, mallable, strong, stiff and light. Composites made with synthetic fillers such as glass or carbon fibres are extensively used for many applications, such as sport, automotive and aerospace. Their success is due to their specific properties, based on a strong interaction between the different components and a great stability.

The strength of a composite material will depend on the strength of the reinforcement component and its interaction with the matrix component. A weak mechanical interaction between the reinforcement component and the matrix component results in a composite material with limited practical applications. Improving the strength of the reinforcement component and the interaction of the reinforcement and the matrix components therefore provides composite materials which are stronger, more durable and less susceptible to stress and wear.

Cellulose or plant fibres have been used in some applications in the art as reinforcement agents, such as the manufacture of paper. There are a number of sources of cellulose fibres. Cellulose microfibrils can be extracted from wood pulp or cotton however, pulping and bleaching processes are not environmentally friendly.

Cellulose whiskers called tunicin can also be extracted from tunicate, a sea animal. Finally, bacterial cellulose or nanocellulose can be produced by specific bacteria strains, the most efficient producer being Acetobacter xylinum.

The present invention provides surface modified polymers which can be used as reinforcement or matrix components in composite materials.

The first aspect of the present invention therefore provides a material comprising cellulose associated with a support selected from a polymer and/or a fibre, wherein said cellulose is produced by a micro-organism.

The cellulose is preferably linked to the support. The cellulose can be linked to the support by covalent bonding, hydrogen bonds, electrostatic interactions, by the formation of a crystalline layer (such as a transcrystalline layer) and/or van der Waals interaction. In a preferred aspect of the invention, the material of the first aspect is produced by culturing the support in the presence of the cellulose producing micro-organisms. To this end, the cellulose is preferably produced by the cellulose producing micro-organism directly onto the support. The micro-organism produced cellulose is therefore preferably strongly attached to the support. Alternatively, the cellulose may be produced in a micro-organism culture and subsequently attached to the support by post-synthesis modification.

The first aspect of the invention therefore preferably provides a material comprising cellulose associated with a support selected from a polymer and/or a fibre wherein the material is produced by culturing the support with a cellulose producing micro-organism.

The association of the cellulose with the support results in the reinforcement of the support and an increase in the surface area of the support. When the material is provided as a reinforcement and/or a matrix for a composite material, the increased surface area of the support provides enhanced adhesion properties and allows an improved interaction between the reinforcement and the matrix. The material of the first aspect therefore exhibits increased tensile strength and elastic modulus. The support is provided as a polymer or a fibre or a mixture thereof. In particular, the support can be provided as a pellet, a powder, loose fibres, a woven or non-woven fibre mat, a string or a tow. The polymer or fibre are preferably a reinforcement component or matrix component as used in the art for the manufacture of composite materials When the support is a polymer, it is preferably provided in the form of a fibre, pellet or a powder. The polymer can be a synthetic polymer or a naturally derived or occurring polymer. Where the polymer is a synthetic polymer, it can be a plastic or can be an oil-based polymer such as polyethylene, polypropylene or polymethylmethacrylate. Alternatively, the polymer is an oil based polymer having a processing temperature below the degradation temperature of cellulose. The polymer can be a synthetic bioderived polymer such as poly(lactic acid), polyhydroxyalkanoate (PHA, bacterial poly esters) or modified cellulose polymers such as cellulose acetate butyrate (CAB) or cellulose butyrate. The polymer can be a naturally occurring polymer such as wheat gluten, corn zein, wool, cellulose or starch. When the support is a fibre, it can be a glass or carbon based fibre. The fibre can be derived or obtained from a plant or animal. In particular, the fibre is preferably extracted from a plant, such as one or more of abaca, bamboo, banana, coir, coconut husk, cotton, flax, henequen, hemp, hop, jute, palm, ramie or sisal. Where the support is obtained or derived from a natural source, the support can be biodegradable. It will be appreciated that the provision of a reinforced biodegradable material will provide benefits, particularly when used in composite materials. In particular, conventionally used glass or carbon fibre composites are very difficult to recycle. The provision of biodegradable composite materials would therefore overcome the end-of-life disposal problems associated with conventional composite materials. This is particularly important in light of current European waste legislation (such as the landfill directive 1999/31/EC, the End-of-Life vehicle directive 2000/53/EC and the Waste Electrical and Electronic Equipment Directive 2002/96/EC) which bans the use of landfill (or makes landfill disposal prohibitively expensive) in most EU member states.

The support is reinforced by cellulose which is associated with or attached to the support. The cellulose is produced by a micro-organism, preferably by a bacteria. The shape and size of the cellulose will depend on the micro-organism. The cellulose is preferably produced as a nanofibre, such as a ribbon shaped nanofibril. The cellulose nanofibre preferably has a thickness of from 0.5 to 50 nm, preferably from 1 to 20 nm, more preferably from 2 to 10 nm, most preferably 1, 2, 3, 4, 5, 6, 7, 8, 9 or 10 nm. The cellulose fibre preferably has a width of from 0.5 to 100 nm, preferably 1 to 50 nm, more preferably 5 to 20 nm. The cellulose fibre preferably has a length of 0.5 micrometres to 1000 micrometres, preferably 1 micrometres to 500 micrometres, more preferably 5 to 300 micrometres, most preferably 10 to 150 micrometres.

The second aspect of the invention relates to a process for the production of a material of the first aspect of the invention comprising contacting a culture medium comprising a support selected from a polymer or a fibre with a cellulose producing micro-organism.

For the purposes of the second aspect of the invention, the cellulose producing micro-organism can belong to the genera, Acetobacter, Rhizobium, Alcaligenes, Agrobacterium, Sarcina and/or Pseudomonas.

In a preferred feature of the second aspect the micro-organism is a strain adapted to culture in agitated conditions, such as Acetobacter xylinum BPR2001.

In a preferred feature of the second aspect of the invention, the support can be modified by physical or chemical treatments prior to incubation with a cellulose producing micro-organism, such as atmospheric or low pressure plasma or corona treatments, solvent washing or extraction, bleaching, boiling or washing, for example in a basic solution, such as sodium hydroxide solution. In particular, the support can be washed with a solvent, such as an organic solvent (i.e. acetone, ethyl acetate etc. or an alcohol such as ethanol, methanol, propanol, butanol etc.) prior to incubation with the cellulose producing micro-organism.

The culture medium is preferably incubated in the presence of the cellulose producing micro-organism with agitation. In particular, incubation can occur in a shake flask, in a fermentor or a rotating-disk bioreactor. Preferably the incubation is carried out such that the support is periodically or continuously in contact with culture medium and air.

The material is produced by the production of cellulose by a micro-organism on a support. The microorganisms producing cellulose can be cultured in a medium comprising the support. The micro-organisms grow, preferably on the support surface rather than freely in the medium, such that the produced cellulose is strongly attached to the support. The material can then be used as reinforcing agent in a matrix in order to create a fully hierarchical composite.

The material can be further reinforced by the association of the material with free cellulose, either present in the medium or added to the medium. The association of the material with the free cellulose is achieved by heat treatment, chemical modification of the material, etc.

The culture medium comprises a carbon source, a nitrogen source, inorganic salts and preferably trace nutrients such as amino acids and vitamins. The support can be provided in the culture medium in the form of pellets, a powder, loose fibres, a woven or non-woven fibre mat, a string or a tow.

In a particularly preferred feature of the second aspect, the support is autoclaved with the medium in the flask, fermentor or bioreactor. The medium is then inoculated with the cellulose producing micro-organism (preferably as a two or three days old previous culture broth comprising the cellulose producing micro-organism) and incubated at 25 to 35° C., preferably 28 to 30° C., more preferably 28, 29 or 30° C. The culture medium is preferably regulated at a pH of from 4 to 7, more preferably at a pH of from 5 to 6. The medium is preferably supplied with air to ensure aerobic conditions for bacterial growth. Incubation is usually carried out for a period of days to several weeks, such as 3 days to 1 week, preferably 3, 4, 5, 6 or 7 days, usually until one nutrient in the culture medium is consumed.

The modified material is then harvested and either used directly or is purified in basic conditions (for example NaOH, K₂CO₃, KOH etc with heating, such as 0.1 N NaOH at 80° C. for 20 min) in order to remove all microorganisms. If the material has been purified under basic conditions, the modified material can then be thoroughly washed with distilled water until neutral pH. The modified material can be stored at room temperature and pressure.

In an alternative feature of the second aspect of the invention, the cellulose can be produced by the cellulose producing micro-organism and then associated with the support. The cellulose can be associated with the support by chemical modification of the support, heat treatment or any other method known in the art.

The third aspect of the invention relates to a composite material comprising a reinforcement and a matrix wherein the reinforcement and/or the matrix comprises a material of the first aspect of the invention. The composite material of the third aspect is a cellulose nanocomposite.

The material of the first aspect can be used as a reinforcing agent for composite manufacturing. The material can therefore be combined with any conventional matrix known to a person skilled in the art. Where the material is biodegradable, in order to preserve the renewability and biodegradability of the material, bioderived polymers such as poly(lactic acid) (PLA), polyhydroxyalkanoates (PHA, bacterial polyesters), or modified cellulose polymers (cellulose acetate butyrate (CAB) or cellulose butyrate), as well as plant based resins can be used as a matrix.

Alternatively, the material of the first aspect can be used as a matrix for composite manufacturing. The material can therefore be combined with any conventional reinforcement known to a person skilled in the art. Where the matrix is biodegradable, in order to preserve the renewability and biodegradability of the material, the matrix can be combined with a biodegradable reinforcement material.

The fourth aspect of the invention relates to a process for the production of a composite material according to the third aspect of the invention wherein the support of the first aspect is impregnated, mixed or extruded with a polymer/resin. The composite can be manufactured using any suitable process such as resin transfer moulding, sheet moulding, resin infusion moulding, or by powder impregnation and compression moulding.

The fifth aspect of the invention relates to an article produced from the composite material of the third aspect of the invention. The composite material is particularly provided for use in low-load applications, including but not limited to packaging, or use in the automotive, household, sport and/or construction industries. The article of the fifth aspect is preferably produced from a fully biodegradable composite material.

All preferred features of each of the aspects of the invention apply to all other aspects inutatis mutandis.

The invention may be put into practice in various ways and a number of specific embodiments will be described by way of example to illustrate the invention with reference to the accompanying drawings, in which:

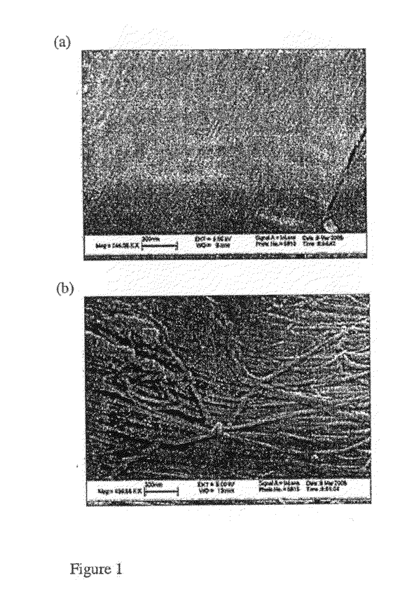

FIG. 1: SEM pictures of the surface of an hemp fibre from (a) an untreated hemp mat and from (b) an hemp mat on which bacterial cellulose was grown.

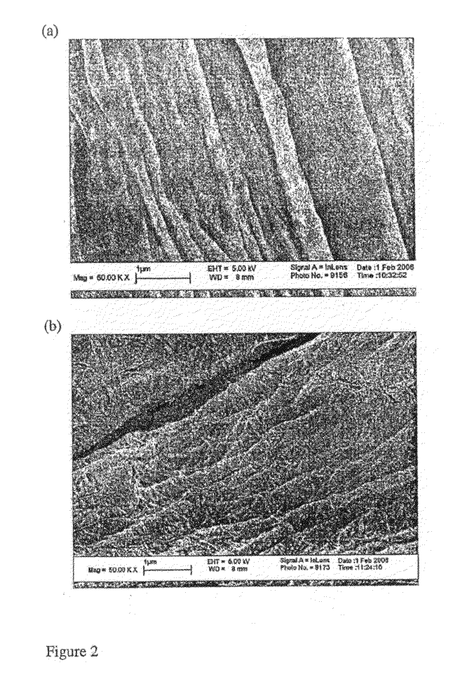

FIG. 2: SEM pictures of the surface of a loose hemp fibre stayed in the medium without (a) or with (b) bacteria inoculation and that was further treated with NaOH.

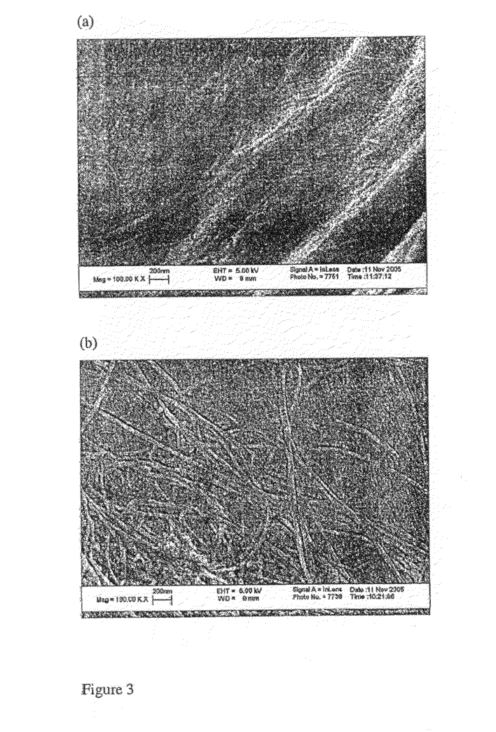

FIG. 3: SEM pictures of the surface of a loose sisal fibre stayed in the medium without (a) or with (b) bacteria.

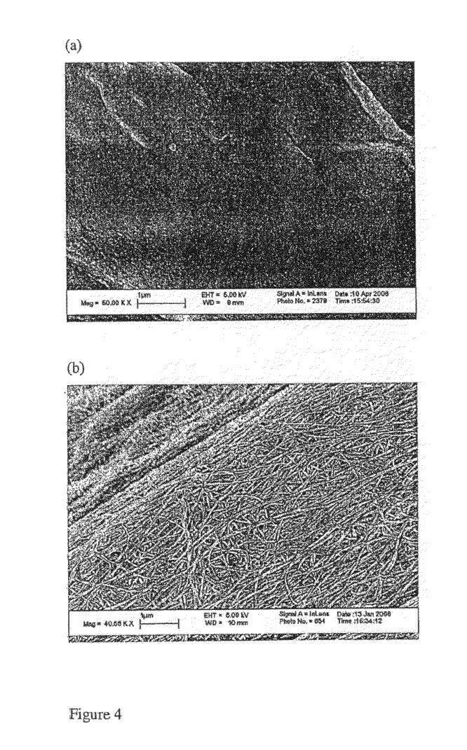

FIG. 4: SEM pictures of the surface of a loose sisal fibre washed with acetone (a) and after that bacterial cellulose was grown on its surface (b).

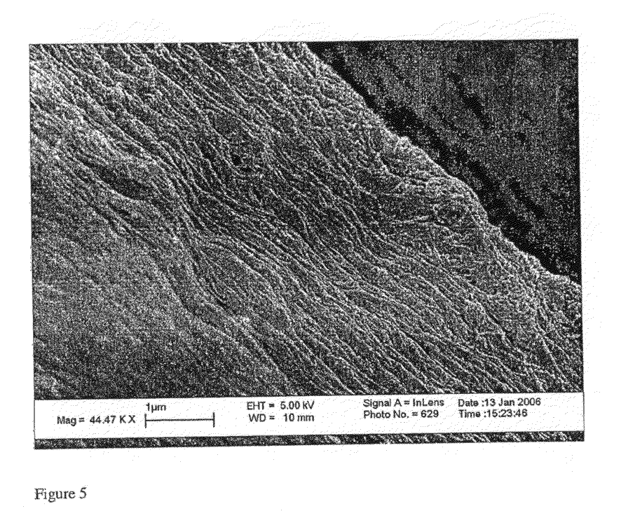

FIG. 5: Scanning electron micrograph (SEM) of modified jute.

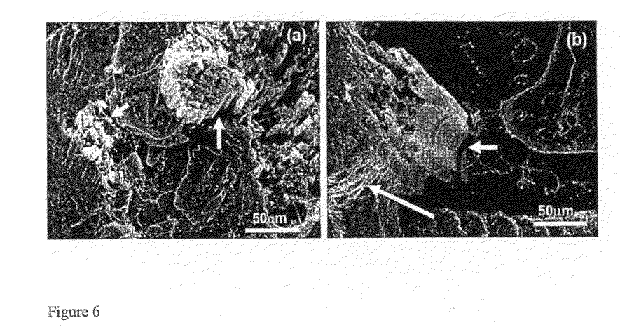

FIG. 6: SEM micrographs of composite fracture surfaces: (a) unmodified sisal and (b) modified sisal in poly-L-lactic acid matrix

The present invention will now be illustrated by reference to one or more of the following non-limiting examples.

### EXAMPLES

### Example 1

One piece of hemp mat (4×4 cm) was put in a 250 ml Erlenmeyer flask filled with 75 ml of medium made of 50 g/l glucose, 5 g/l yeast extract and 12.5 g/l calcium carbonate. The flask was inoculated with 3 ml of a three days old broth of a previous culture of Acetobacter xylinum BPR2001. The culture was conducted on a shaking plate, in a chamber at 30° C. The pH was not regulated. The hemp mat was removed after 3 days.

Some fibres situated in the middle of the mat were collected and washed with acetic acid to remove the calcium carbonate. After drying, scanning electron microscopy (SEM) analysis was performed both on these fibres and on dried hemp fibres from an untreated hemp mat (FIG. 1). Bacterial cellulose nanofibres can be observed attached to the hemp fibre surface, even after acetic acid treatment. The surface area of the modified fibre is consequently increased. This increase in surface area will lead to a significant increase in the practical adhesion of this modified fibre to the matrix, when using it as a reinforcement in a composite material.

### Example 2

Loose hemp and sisal plant fibres (0.5 g each) were separately put in 250 ml Erlenmeyer flasks containing 90 ml of a medium made of 50 g/l fructose, 5 g/l yeast extract, 2.7 g/l Na₂HPO₄ and 1.15 g/l citric acid. The autoclaved flasks were inoculated with 10 ml of a three days old previous culture of Acetobacter xylinum BPR2001. The culture was conducted on a shaking plate, in a chamber at 30° C. The pH was not regulated and the fibres were removed after one week. Some fibres were further treated with NaOH 0.1M at 80° C. for 20 min, then thoroughly washed with deionised water.

SEM analysis reveals that part of the surface of sisal fibres and the whole surface of hemp fibres incubated with the bacteria were completely covered with bacterial cellulose nanofibres, even after NaOH extraction (FIGS. 2 and 3). A dramatic increase in the surface area of the modified fibres can consequently be expected. Tensile testing (according to ASTM D 3379-75) was carried out on the fibres (natural ones and those incubated with the bacteria). Results show that the mechanical performance of the sisal fibres was not affected by the incubation in the medium, nor by the NaOH treatment (Table 1). Regarding hemp fibres (Table 2), a decrease in strength and stiffness can be observed, but it is most probably due to a splitting phenomenon of the fibres when drying. The NaOH treatment has no effect on the mechanical performance of the fibres.

TABLE 1

| | Young's | Tensile | Elongation |
|---|---|---|---|
| | modulus | strength | at break |
| Sample | (GPa) | (MPa) | (%) |
| 1: Unmodified natural sisal fibre | 15.0 (±3.8) | 342 (±105) | 2.9 (±0.3) |
| 2: Natural sisal fibre in medium | 13.8 (±5.0) | 352 (±118) | 5.4 (±2.8) |
| without bacteria | | | |
| 3: Sample 2 with further NaOH | 12.2 (±4.0) | 343 (±67) | 4.8 (±1.9) |
| extraction | | | |
| 4: Natural sisal fibre in medium | 12.5 (±2.9) | 324 (±100) | 4.5 (±1.1) |
| with bacteria (fibre on which | | | |
| bacterial cellulose was grown) | | | |
| 5: Sample 4 with further NaOH | 11.7 (±2.8) | 285 (±65) | 4.3 (±1.5) |
| extraction | | | |

TABLE 2

| | Young's | Tensile | Elongation |
|---|---|---|---|
| | modulus | strength | at break |
| Sample | (GPa) | (MPa) | (%) |
| 1: Unmodified natural hemp | 21.4 (±4.8) | 286 (±76) | 2.0 (±0.6) |
| fibre | | | |
| 2: Natural hemp fibre in medium | 13.5 (±7.6) | 263 (±61) | 2.7 (±0.6) |
| without bacteria | | | |
| 3: Sample 2 with further NaOH | 15.1 (±4.8) | 224 (±109) | 2.5 (±0.7) |
| extraction | | | |
| 4: Natural hemp fibre in medium | 8.8 (±1.9) | 171 (±30) | 2.9 (±0.7) |
| with bacteria (fibre on which | | | |
| bacterial cellulose was grown) | | | |
| 5: Sample 4 with further NaOH | 8.0 (±1.5) | 130 (±33) | 2.3 (±0.5) |
| extraction | | | |

The adhesion between the fibres (natural and modified) and a potential polymeric matrix (cellulose acetate butyrate CAB) was assessed by a single fibre pull out test. The resulted interfacial shear strength (IBS) are given in the Table 3. In both types of fibres, the presence of bacterial cellulose at their surface has enhanced the adhesion between the fibre and the matrix from a factor of 1.5 to 2.4.

TABLE 3

| Sample | IFSS (MPa) |
|---|---|
| Natural sisal fibre | 1.02 (±0.23) |
| Sisal fibre on which bacterial cellulose was grown | 1.49 (±0.06) |
| Natural hemp fibre | 0.76 (±0.18) |
| Hemp fibre on which bacterial cellulose was grown | 1.83 (±0.34) |

### Example 3

Sisal loose fibres were washed with acetone before being put in a 250 ml Erlenmeyer flask containing 90 ml of a medium made of 50 g/l fructose, 5 g/l yeast extract, 2.7 g/l Na₂HPO₄ and 1.15 g/l citric acid. The autoclaved flask was inoculated with 10 ml of a three days old previous culture of Acetobacter xylinum BPR2001. The culture was conducted on a shaking plate, in a chamber at 30° C. The pH was not regulated and the fibres were removed after one week.

SEM pictures of the surface of the fibres, before and after bacterial cellulose growing are shown on FIG. 4. The preliminary acetone treatment of the fibres significantly improved the growing content of the bacterial cellulose on the surface of the fibres since it was found to be entirely covered by bacterial cellulose.

### Example 4

### Application of the Bacterial Cellulose Attaching Technique Upon Plants Fibre Other than Hemp And Sisal

The technique for application of the bacterial cellulose has been applied to other plant fibres, namely jute, flax, bamboo, abaca and ramie. It can be observed that after the fibres have been in the bacterial broth for 1 week a white gel layer of bacterial cellulose appeared and covered the surface of all fibres. However, this layer was smaller in the case of abaca fibres.

The fibres were treated with acetone (ethanol is also possible) to remove the waxy substances attached to original natural fibres before the bacterial culture. The removal of the hydrophobic waxy layer promotes the compatibility between the bacteria and the plant fibre surface and in turn promotes the deposition of bacterial cellulose around the fibre leading to a higher surface coverage of the plant fibre.

FIG. 5 provides a scanning electron micrograph (SEM) of modified jute.

### Example 5

### Improvement in Interfacial Adhesion Between the Modified Fibres and the Bio-Based Polymers

The interfacial adhesion between the modified plant fibres and bio-based polymer matrices was found to increase significantly after deposition of bacterial cellulose around the fibres. As measure of the interfacial adhesion the interfacial shear strength (IFSS) was measured by the single fibre pullout test. (Modified) Sisal and hemp fibres were embedded in to two bio-based polymers; poly-L-lactic acid (PLLA) and cellulose acetate butyrate (CAB). The results are shown in Table 4.

TABLE 4
Interfacial shear strength (IFSS) of fibres in
bio-based polymer matrices

| Treatment | IFSS/MPa |
|---|---|
| Unmodified Sisal in | 12.10 ± 0.46 |
| PLLA | |
| Modified Sisal in PLLA | 14.55 ± 1.20 |
| Unmodified Sisal in CAB | 1.02 ± 0.43 |
| Modified Sisal in CAB | 1.49 ± 0.02 |
| Unmodified Hemp in | 0.76 ± 0.32 |
| CAB | |
| Modified Hemp in CAB | 1.83 ± 0.27 |

The improved interfacial adhesion was also confirmed by the SEM of the cryogenically-fractured composites. The composites contain original unmodified sisal and sisal modified by the deposition of bacterial cellulose. FIG. 6 shows the fracture of composites fabricated with PLLA and unmodified sisal (FIG. 6a), and modified sisal (FIG. 6b). It can be observed that in the case of unmodified sisal, there is a gap between the fibre and the matrix, as indicated by the arrow. In the case of the modified fibre, despite some gap remains at the interface (short arrow), some ‘wetting’ of the polymer on the fibre can also be observed (long arrow), indicating the improved compatibility.

It is understood that the improvement in the compatibility is due to the chemical bonding between the two components. Bacterial cellulose contains a high number of hydroxyl groups, which have the potential to form hydrogen bonds to the polymer functional groups. The presence of the highly-crystalline bacterial cellulose can also lead to the formation of a transcrystalline layer of the matrix, which further strengthens the adhesion between the fibre and the matrix.

### Example 6

### Improved Performance of the Composites Fabricated with Modified Fibres

Preparation of unidirectional Long Fibre Reinforced Composites

Unidirectional long natural fibre reinforced composites were prepared with fibres aligned at 0° and 90° to the test direction. The fibres were impregnated by polymer powder using a dusting sieve. The impregnated fibres were then clamped at both ends of a metal mould prior to compression moulding, to ensure a high degree of alignment in the final composite tape. The fibre content of the natural fibre reinforced composites was adjusted to 34% by weight. The clamped impregnated fibres were then compression-moulded in a′ hot press (George E Moore & Sons, Birmingham, UK) at 195° C. (for CAB) and 220° C. (for PLLA) and 1.8 MPa for 5 min, and left to cool down under load at a rate of approximately 4° C./min. The 0° composite tapes had dimensions 150×12.5×1 mm whilst the 90° composite tapes were 100×120×1 MM.

Unidirectional continuous fibre reinforced bio-based polymer composites were fabricated and tested in the direction parallel to the fibre alignment (0°. It was found that with 34% wt sisal reinforcement in PLLA matrix, the tensile strength improved by 44% while the Young's modulus improved by 42% (Table 5) with the modified sisal. This improvement in the tensile property is a direct effect from the improved adhesion between the fibre and the matrix, since the stress is able to transfer from the matrix to the fibre better. The presence of strong nano-size bacterial cellulose helps to reinforce the composites.

In addition, composites fabricated with modified fibres were found to absorb less water. After 1 week of immersion in water at room temperature, composites fabricated with unmodified sisal absorb water up to 50%, which is 13% more than the composites fabricated with modified fibres. The water absorption rate for the unmodified sisal composites is also higher (Table 5). The reduction in the water uptake is understood to result from the improved adhesion between the fibre and the matrix. Since there is less gap at the interface, this lessens the water holding capability of the composites.

TABLE 5
Tensile property and water absorption parameters of
composites fabricated with 34% wt unmodified sisal
and modified sisal

| | Tensile | Young's | Water | Water |
|---|---|---|---|---|
| | Strength, | Modulus, | Absorption | Uptake/ |
| Sisal Fibre | MPa | GPa | Rate, % h^{-1/2} | % |
| Unmodified Sisal | 78.9 ± 8.5 | 7.91 ± 0.77 | 33.9 ± 1.0 | 50.0 ± 2.8 |
| Modified Sisal | 113.8 ± 8.1 | 11.21 ± 0.69 | 28.9 ± 0.3 | 36.6 ± 0.4 |

### Example 7

### Short Fibre Composites

CAB and PLLA polymer are mixed with sisal or hemp fibres with Brabender Mixer W 50 EHT (Brabender® GmbH & Co. KG, Germany) at 15° C. above the polymer melting point for 5 min. The mixture is then hot moulded at the same temperature under 1.5 MPa pressure for 5 min before cooled down at approximately 5° C. per min under pressure to obtain the 1 mm composite film. The modified sisal lead to the improvement in 20% wt sisal reinforced CAB, as shown in Table 6.

TABLE 6
Tensile property of 20% wt sisal reinforced CAB composites

| | Tensile | Young's |
|---|---|---|
| Sisal Fibre | Strength, MPa | Modulus, GPa |
| Unmodified | 22.2 ± 0.7 | 1.86 ± 0.04 |
| Sisal | | |
| Modified Sisal | 24.7 ± 1.4 | 1.90 ± 0.05 |

The observed improvement originates from the improved adhesion between the fibre and the matrix, and the direct reinforcement of the bacterial cellulose.

1. A material comprising cellulose associated with a support selected from a polymer or a fibre, wherein said cellulose is produced by a micro-organism.

2. The material as claimed in claim 1 wherein the material is produced by culturing the support with a cellulose producing micro-organism.

3. The material as claimed in claim 1 wherein the cellulose is associated with the support by covalent bonding, hydrogen bonding, electrostatic interactions or van der Waals interactions.

4. The material as claimed in claim 1 wherein the support is obtainable from a natural or synthetic source.

5. The material as claimed in claim 1 wherein the support is a polymer selected from one or more of an oil-based polymer, a synthetic bioderived polymer, a naturally occurring polymer, and combinations thereof.

6. The material as claimed in claim 5 wherein the oil based polymer is selected from one or more of polyethylene, polypropylene, or polymethylmethacrylate.

7. The material as claimed in claim 5 wherein the synthetic bioderived polymer is selected from one or more of-poly(lactic acid), polyhydroxyalkanoate (PHA, bacterial poly esters) cellulose acetate butyrate (CAB) or cellulose butyrate.

8. The material as claimed in claim 5 wherein the naturally occurring polymer is selected from one or more of-wheat gluten, corn zein, wool, cellulose or starch.

9. The material as claimed in claim 1 wherein the support is a fibre selected from one or more of a glass based fibre, a carbon based fibre, a plant fibre or an animal fibre.

10. The material as claimed in claim 9 wherein the plant fibre is derived or obtained from one or more of abaca, bamboo, banana, coir, coconut husk, cotton, flax, henequen, hemp, hop, jute, palm, ramie or sisal.

11. The material as claimed in claim 1 wherein the support is biodegradable.

12. A process for the production of a material as claimed in claim 1 comprising contacting a culture medium comprising a support selected from a polymer or a fibre with a cellulose producing micro-organism.

13. The process as claimed in claim 12 wherein the support is further incubated with cellulose produced by a cellulose producing micro-organism, such that the cellulose is associated with the support.

14. The process as claimed in claim 13 wherein the cellulose produced by a cellulose producing micro-organism is present in the culture medium or is added to the culture medium.

15. The process as claimed in claim 12 wherein the cellulose producing micro-organism is selected from Acetobacter, Rhizobium, Alcaligenes, Agrobacterium, Sarcina, or Pseudomonas.

16. The process as claimed in claim 12 wherein the support is provided in the form of pellets, a powder, loose fibres, a woven or non-woven fibre mat, a stringor a tow.

17. The process as claimed in claim 12 wherein the support is incubated with the cellulose producing micro-organism with agitation.

18. The process as claimed in claim 12 wherein the support is modified by a physical or chemical treatment prior to treatment with the cellulose producing micro-organism.

19. A process for the production of a material as claimed in of claim 1 comprising incubating a support selected from a polymer or a fibre with cellulose produced by a cellulose producing micro-organism, such that the cellulose associates with the support.

20. The process as claimed in claim 19 wherein the cellulose is associated with the support by chemical modification of the support or heat treatment.

21. (canceled)

22. A process for the production of a composite material as claimed in claim 5 wherein reinforcement comprises a material as claimed in claim 1, and said reinforcement is impregnated, mixed or extruded with a matrix.

23.-26. (canceled)

27. The material of claim 10 wherein the plant fibre is hemp and the micro-organism is Acetobacter.

28. The material of claim 10 wherein the plant fibre is sisal and the micro-organism is Acetobacter.

29. The material of claim 1 wherein the support is a composite comprising a reinforcement fibre and a matrix.

30. The material of claim 29 wherein the reinforcement fibre is selected from abaca, bamboo, banana, coir, coconut husk, cotton, flax, henequen, hemp, hop, jute, palm, ramie, sisal and combinations thereof and the matrix is selected from poly(lactic acid), polyhydroxyalkanoate (PHA, bacterial poly esters) cellulose acetate butyrate (CAB), cellulose butyrate, and combinations thereof.

31. The material of claim 31 wherein the reinforcement fibre is hemp or sisal, the matrix is poly(lactic acid) or cellulose acetate butyrate and the micro-organism is Acetobacter.
